(19) United States
(12) Patent Application Publication
(10) Pub. No.: US 2011/0009522 A1
(43) Pub. Date: Jan. 13, 2011

(54) MATERIAL FOR FILLING BONE DEFECTS AND PRODUCTION METHOD THEREOF

(75) Inventors: Toshihiro KASUGA, Kiyosu (JP); Akiko OBATA, Nagoya (JP); Kie FUJIKURA, Nagoya (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA INSTITUTE OF TECHNOLOGY, NAGOYA-SHI (JP)

(21) Appl. No.: 12/707,302

(22) Filed: Feb. 17, 2010

(30) Foreign Application Priority Data
Jul. 10, 2009 (JP) 2009-163320

(51) Int. Cl. A61F 2/28; B29C 70/00

(52) U.S. Cl. 523/116; 264/484

(57) ABSTRACT

Disclosed is a material for filling bone defects having a three-dimensional steric structure. This material is produced by dissolving or suspending a substance in a solvent to give a solution or slurry, the substance containing a biodegradable resin as a principal component and bearing a siloxane; adding water to the solution or slurry to give a spinning solution, the water having a relative dielectric constant larger than that of the biodegradable resin; subjecting the spinning solution to electrospinning while applying a positive charge to a collector by a voltage supply and grounding a nozzle of a syringe without applying a charge thereto; thereby yielding the material on the collector.

### CROSS REFERENCE TO RELATED APPLICATION

This application is based upon and claims the benefit of Japanese Patent Application No. 2009-163320 filed on Jul. 10, 2009, the content of which are incorporated herein by reference.

### FIELD OF THE INVENTION

The present invention relates to bioactive materials which are useful as bone-repairing materials for filling bone defects and are used in the fields of oral or maxillofacial surgery and orthopedic surgery. More specifically, the present invention relates to a material for filling bone defects, which material has a three-dimensional steric structure including, as its skeleton, a composite fiber with a bioresorbable-biodegradable resin. Such a bioresorbable-biodegradable resin helps to improve the affinity for the bone and can be absorbed in vivo. The present invention also relates to a method for producing the material for filling bone defects.

### RELATED ART OF THE INVENTION

Some materials, when buried or implanted in bone defects, react with the bone and are directly chemically combined with the bone. These materials are called bioactive materials and are further classified into superficial bioactive materials, where the reaction occurs only in the surface of materials; and bioresorbable materials, where the reaction occurs even inside of materials and the materials are gradually replaced with the bone. Exemplary commercialized superficial bioactive materials include hydroxyapatite ceramics (e.g., trade name APACERAM supplied by HOYA CORPORATION, Japan); and exemplary commercialized bioresorbable materials include beta phase tricalcium phosphate ceramics (e.g., trade name OSferion supplied by Olympus Terumo Biomaterials Corp., Japan).

Calcium carbonate (CaCO₃) and gypsum (CaSO₄·2H₂O) are also known to be bioresorbable. These substances, however, have low strength and toughness and are difficult to be machined. In contrast, biodegradable polymers such as poly(lactic acid)s, poly(glycolic acid)s, copolymers of them, and polycaprolactones are highly flexible and are easy to be machined. The biodegradable polymers, however, do not show osteogenic ability (bone forming ability) because their biodegradability is derived from the phenomenon that they are degraded in vivo and are discharged therefrom. In addition, there have been some reports that some of the biodegradable polymers may affect surrounding tissues because they are degraded typically into lactic acid or glycolic acid upon degradation and thus show acidity. Under such circumstances, there have been made investigations to provide composite materials between these inorganic compounds and organic compounds to allow the composite materials to have both osteogenic ability and bioresorbability and further have improved mechanical properties. Typically, Japanese Unexamined Patent Application Publication (JP-A) No. 2001-294673 discloses a process for the preparation of a bioresorbable material by combining a poly (lactic acid) and a calcium. carbonate. Specifically, this document refers to a process for synthesizing a bioresorbable material by mixing a calcium carbonate containing vaterite as a principal component with a biodegradable polymer compound such as a poly (lactic acid), which vaterite is highly soluble in water among such calcium carbonates. This technique is also advantageous in that the pH is always maintained around neutrality, because even when the poly (lactic acid) is decomposed to be acidic, the acidity is neutralized by the buffering effects of the calcium carbonate as dissolved.

In this unprecedented aged society, bone defects should be desirably cured as soon as possible, because it is very important to maintain and ensure mastication and exercise performance for the health maintenance. To improve osteogenic ability, there have been attempted to incorporate, to a bioresorbable membrane, a factor such as an bone formation inducer (see Japanese Unexamined Patent Application Publication (JP-A) No. H06 (1994) -319794), or a proliferation factor or a bone morphogenic protein (see Japanese Unexamined Patent Application Publication (Translation of PCT Application) (JP-A) No. 2001-519210; and Japanese Unexamined Patent Application Publication (JP-A) No. 2006-187303). However, it is difficult to handle these factors. Accordingly, demands have been made to develop a bioresorbable material having superior bone reconstruction ability to allow the bone to self-regenerate more reliably and more rapidly.

In view of recent trends of researches and technologies for bio-related materials, the main stream of researches has been shifted from a materials design for the bonding of a material with the bone to a materials design for the regeneration of the bone; in these researches, the role of silicon in bone formation has been received attention; and there have been designed a variety of silicon-doped materials (TSURU Kanji, OGAWA Tetsuro, and OGUSHI Hajime, “Recent Trends of Bioceramics Research, Technology and Standardization”, Ceramics Japan, 41, 549-553 (2006)). For example, there has been reported that the controlled release of silicon genetically acts on cells to promote bone formation (H. Maeda, T. Kasuga, and L. L. Hench, “Preparation of Poly(L-lactic acid)-Polysiloxane-Calcium Carbonate Hybrid Membranes for Guided Bone Regeneration”, Biomaterials, 27, 1216-1222 (2006)). Independently, when composites of a poly(lactic acid) with one of three calcium carbonates (calcite, aragonite, and vaterite) are prepared and soaked in a simulated body fluid (SBF), the composite of the poly(lactic acid) with vaterite forms a hydroxyapatite having bone-like composition and dimensions within a shortest time among the three composites (H. Maeda, T. Kasuga, M. Nogami, and Y Ota, “ Preparation of Calcium Carbonate Composite and Their Apatite-Forming Ability in Simulated Body Fluid”, J. Ceram. Soc. Japan, 112, S804-808 (2004)). These findings demonstrate that the use of vaterite which gradually releases silicon is believed to be a key to provide a material that gives more rapid bone reconstruction.

To use a material for filling bone defects, the affected area (bone defect) is incised, and a dense or porous material having such a size as to fill the affected area sufficiently is directly implanted, or a granular material is charged into the affected area.

For ensuring bone formation, it is desirable to implant or bury such a material in the affected area without a gap (clearance). However, it is not easy to process a dense or porous material so as to fit the dimensions of the affected area; and a granular material, if charged, often drops off from the affected area after the surgery (implantation). These techniques are therefore susceptible to improvements.

Independently, though being not a technique of charging such a material into the affected area, there is also known a guided bone regeneration technique of using a masking membrane to cover a bone defect. The masking membrane has the functions of preventing the invasion of cells and tissues not involved in bone formation into the bone defect, allowing the self-regeneration ability of the bone to exhibit, and helping the bone to reconstruct. This technique is intended to cure the bone defect by using the curing ability which a living body inherently has. For example, Japanese Unexamined Patent Application Publication (JP-A) No. 2009-61109 discloses a guided bone regeneration membrane and a production method thereof, which guided bone regeneration membrane has a bi-layer structure including a first nonwoven fabric layer and a second nonwoven fabric layer, in which the first nonwoven fabric layer contains a silicon-releasable calcium carbonate and a biodegradable resin as principal components, and the second nonwoven fabric layer contains a biodegradable resin as a principal component. It has been reported that the use of this membrane gives satisfactory proliferation of murine osteoblast-like cells (MC3T3-E1 cells), and when a bone defect formed in a rabbit cranial bone is covered by the membrane, satisfactory bone formation (osteogenesis) is observed in the membrane (see T. Wakita, A. Obata and T. Kasuga, “New Fabrication Process of Layered Membranes Based on Poly (Lactic Acid) Fibers for Guided Bone Regeneration”, Materials Transactions, 50 [7], 1737-1741 (2009)). This membrane, however, is not usable as a material for filling bone defects because of having a small thickness of from 230 to 300 μm.

### SUMMARY OF THE INVENTION

Accordingly, an object of the present invention is to provide a bioresorbable material for filling bone defects, which material has a sustained release system with such a chemical composition as to guide bone reconstruction ability effectively and has a three-dimensional steric structure having such a flexibility as to fit in an affected area satisfactorily. Another object of the present invention is to provide a production method of the material

Specifically, the present invention provides, in an embodiment, a material for filling bone defects, the material having a flocculent three-dimensional steric structure including a fibrous substance, in which the fibrous substance contains a biodegradable resin as a principal component and contains or bears a siloxane.

The fibrous substance may have a small diameter of 0.05 μm or more and less than 10 μm. The fibrous substance may be coated with a hydroxyapatite on its surface. The biodegradable resin may be a poly (lactic acid) or a copolymer thereof. The fibrous substance may contain the siloxane as dispersed in calcium carbonate microparticles.

The present invention further provides, in another embodiment, a method for producing a material fox filling bone defects. This method includes the steps of preparing a solution or slurry of a substance by dissolving or suspending the substance in a solvent, the substance containing a biodegradable resin as a principal component and containing or bearing a siloxane; and carrying out electrospinning of the solution or slurry while applying a charge not to the solution or slurry but to a collector to thereby yield a material for filling bone defects on the collector, in which the material has a three-dimensional steric structure including a fibrous substance, and the fibrous substance contains the biodegradable resin as a principal component and contains or bears the siloxane.

In the electrospinning, a charge is applied not to the solution or slurry but to the collector, whereby the solution or slurry drawn toward the collector is not charged by itself, and a fibrous substance derived from the solution or slurry three-dimensionally accumulates or deposits on the collector without suffering from electrostatic repulsion. This gives a material for filling bone defects, which material has a flocculent three-dimensional steric structure including a fibrous substance containing a biodegradable resin as a principal component and containing or bearing a siloxane

In this method, a liquid having a relative dielectric constant greater than that of the biodegradable resin may be added to the solution or slurry. This allows the solution or slurry to be more easily drawn toward the collector by the action of polarization.

In an embodiment, the biodegradable resin may be a poly (lactic acid) or a copolymer thereof; the solvent may be chloroform or dichloromethane; and the liquid having a greater relative dielectric constant may be water. In this embodiment, an amphiphilic liquid which is readily miscible with the solvent and with the water is preferably added to the solution or slurry. The amphiphilic liquid may be at least one selected from the group consisting of methanol, ethanol, propanol, and acetone.

In addition, the electrospun material for filling bone defects may be soaked or immersed in a buffer solution being supersaturated with respect to hydroxyapatite. This allows the fibrous substance to be coated with a hydroxyapatite on its surface.

### BRIEF DESCRIPTION OF THE DRAWINGS

Other objects, features and advantages of the present invention will be understood more fully from the following detailed description made with reference to the accompanying drawings.

In the drawings:

FIG. 1 is an explanatory view of a common electrospinning technique;

FIG. 2 is an explanatory view of an electrospinning technique according to an embodiment of the present invention;

FIG. 3 depicts the appearance of a three-dimensional steric structure prepared in Example 1, indicated with 10-mm squares;

FIG. 4 is a scanning electron micrograph (SEM) of fibers constituting the three-dimensional steric structure prepared in Example 1;

FIG. 5 is a graph showing the release amount of silicon ions from a three-dimensional steric structure Si-PLA₁₅, prepared in Example 1, into a cell culture medium;

FIG. 6 is a graph showing the release amount of silicon ions from a three-dimensional steric structure Si-PLA₅₀, prepared in Example 1, into a cell culture medium;

FIG. 7 is a scanning electron micrograph (SEM) of fibers constituting a Si—CaCO₃/PLA three-dimensional steric structure prepared in Example 2;

FIG. 8 is a scanning electron micrograph (SEM) of fibers constituting a Si—CaCO₃/PLA three-dimensional steric structure obtained after soaking, in 1.5 SBF, the Si—CaCO₃/PLA three-dimensional steric structure prepared in Example 2;

FIG. 9 depicts an X-ray diffraction patterns of the Si—CaCO₃/PLA three-dimensional steric structure prepared in Example 2, before and after soaking in 1.5 SBF; and

FIG. 10 is a graph showing the results in cell proliferation testing of a Si—CaCO₃/PLA three-dimensional steric structure coated with a hydroxyapatite prepared in Example 2 and of a comparative sample.

### DETAILED DESCRIPTION OF THE PREFERRED EMBODIMENTS

The present invention will be described further with reference to various embodiments in the drawings.

According to a preferred embodiment of the present invention, a material for filling bone defects is produced through electrospinning, which material has a three-dimensional steric structure including a fibrous substance containing a biodegradable resin as a principal component and containing or bearing a siloxane According to this embodiment, the electrospinning is performed by an original technique. Specifically, in regular or common electrospinning, a charge is applied not to a collector but to a polymer solution. Contrarily, according to this embodiment of the present invention, a voltage is applied in the direction opposite to that in regular electrospinning, namely, the voltage (charge) is applied not to the polymer solution (or slurry) but to the collector, and the polymer solution is grounded. The polymer solution or slurry is sprayed into fibers while applying the voltage in this manner, and the fibers are entangled to form a steric structure. A material for filling bone defects having a three-dimensional steric structure and being satisfactorily flexible can be obtained through the step of carrying out the originally improved electrospinning technique and the step of soaking the electrospun material in a buffer solution being supersaturated with respect to hydroxyapatite.

Preferred examples of the biodegradable resin include a poly(lactic acid) (PLA); and a copolymer of a poly(lactic acid) and a poly(glycolic acid) (PGA) (i.e., lactic acid-glycolic acid copolymer). Examples of biodegradable resins usable herein include synthetic polymers such as polyethylene glycols (PEGs), polycaprolactones (PCLs), PLAs, PGAs, and copolymers of PEG and PCL; and natural polymers such as fibrin, collagen, alginic acid, hyaluronic acid, chitin, and chitosan. Most representatively, a material for filling bone defects may be produced in the following manner. Initially, a solution is prepared by dissolving a PLA in chloroform (CHCl₃) and/or dichloromethane, and an aqueous solution of aminopropyltriethoxysilane (APTES) is added thereto. In this procedure, the weight ratio of PLA:APTES (PLA to APTES) is possibly from 1:0.01 to 1:0.5, but is preferably from 1:0.01 to 1:0.05 (by weight). This is because most of APTES, if added in an excessively large amount, is dissolved out in early stages of soaking in the aqueous solution and thereby not so effective. The PLA has a molecular weight of from about 20×10⁴ to about 30×10⁴ kDa. The concentration of the PLA in the solution is preferably from 4 to 12 percent by weight for satisfactory spinning. For maintaining satisfactory spinning conditions, dimethylformamide and/or methanol may be added to the solution in a proportion of about 50 percent by weight or less relative to chloroform and/or dichloromethane.

To the resulting solution is added a liquid having a greater relative dielectric constant than that of the biodegradable resin to give a spinning solution for the preparation of a three-dimensional steric structure. Typically, when the biodegradable resin is a poly (lactic acid), a liquid having a greater relative dielectric constant than that of lactic acid is added. Examples of the liquid having a greater relative dielectric constant than that of lactic acid (relative dielectric constant: 22.0) include methanol (relative dielectric constant: 32.6), ethanol (relative dielectric constant: 24.6), ethylene glycol (relative dielectric constant: 37.7), 1,2-propanediol (relative dielectric constant: 32.0), 2,3-butanediol, glycerol (relative dielectric constant: 42.5), acetonitrile (relative dielectric constant: 37.5), propionitrile (relative dielectric constant: 29.7), benzonitrile (relative dielectric constant: 25.2), sulfolane (relative dielectric constant: 43.3), and nitromethane (relative dielectric constant : 35.9). Any of these are effective, but most advantageously water (relative dielectric constant: 70 to 80) is used. However, water is immiscible with and completely separated from chloroform and/or dichloromethane used as a solvent for the FLA. To avoid this, an amphiphilic liquid such as methanol, ethanol, propanol, and/or acetone is preferably in coexistence with the solvent and water. Such amphiphilic liquids for use herein are not limited in their relative dielectric constants, as long as being amphiphilic and satisfactorily miscible both with the solvent such as chloroform and/or dichloromethane and with water. Representatively, 0.5 to 5 g of the amphiphilic liquid, such as methanol, ethanol, propanol, and/or acetone, and 0.5 to 3 g of water are added per 1 g of the PLA.

The spinning solution may be further combined with a calcium carbonate to form a slurry (spinning slurry). This helps the speedup (acceleration) of the step of soaking the electrospun article in a buffer solution being supersaturated with respect to hydroxyapatite to form an absorbable hydroxyapatite thereon. The absorbable hydroxyapatite helps to show higher initial cellular adhesion. The amount of the calciumcarbonate is possibly 60 percent by weight or less, because the calcium carbonate, if added in an amount of more than 60 percent by weight, maybe difficult to mix with the solution to give a homogeneous slurry. However, the calcium carbonate, if added in an amount less than 10 percent by weight, may not exhibit its advantageous effects remarkably. The solution or slurry may further include one or more inorganic substances which are usable in vivo without problems. Examples of such inorganic substances include hydroxyapatite, tricalcium phosphate, calcium sulfate, sodium phosphate, sodium hydrogen phosphate, calcium hydrogen phosphate, octacalcium phosphate, tetracalcium phosphate, calcium pyrophosphate, and calcium chloride.

The material for filling bone defects can also be a substance containing a biodegradable resin as a principal component and further containing or bearing a siloxane. This substance is prepared by preparing calcium carbonate microparticles bearing a siloxane dispersed therein (Si—CaCO₃) typically by the method described in Japanese Unexamined Patent Application Publication (JP-A) No. 2008-100878; and mixing 60 percent by weight or less of the Si—CaCO₃ microparticles with PLA. The amount of the Si—CaCO₃ microparticles is preferably from 10 to 60 percent by weight relative to the PLA, as in the calcium carbonate. To uniformly disperse themicroparticles, the substance is preferably prepared by kneading the PLA and Si—CaCO₃ microparticles in predetermined proportions in a heating kneader to give a composite, and dissolving the composite in the solvent to give a spinning solution.

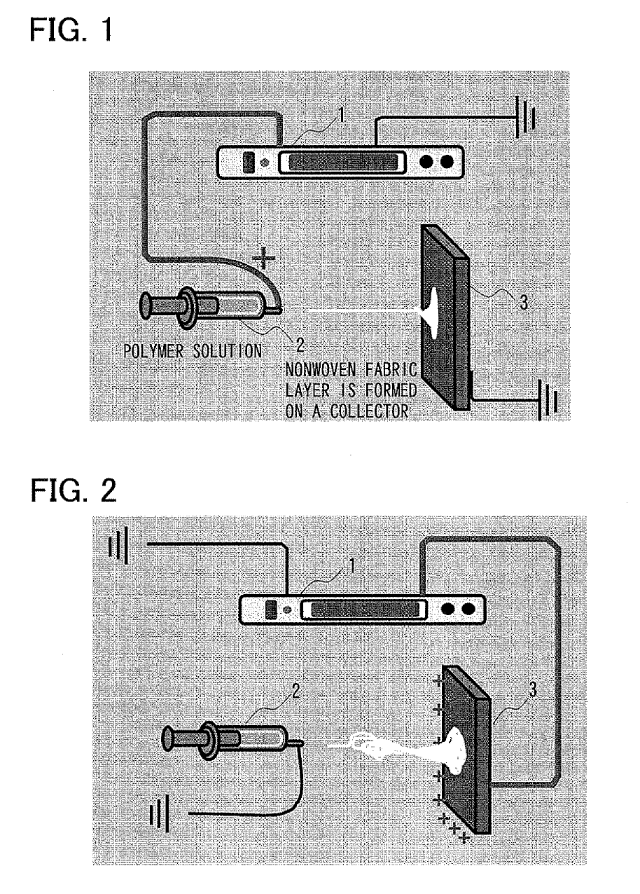

According to a common electrospinning technique with reference to FIG. 1, a charge is applied by a voltage supply 1 to a nozzle of a syringe 2, namely, a positive charge is applied to a spinning solution; and the solution is slowly extruded from the tip of the nozzle. At the time when the effect of electric field becomes larger than the surface tension, the solution is stretched into fibers, travels toward a collector 3 of an earth electrode, reaches the collector 3 while evaporating the solvent, and thereby forms a thin layer of nonwoven fabric of fibers. This technique, however, does not basically give a three-dimensional steric structure even if modifying spinning conditions such as the concentration of the spinning solution, the type of solvent contained in the solution, the supply speed of the solution, the spinning time, the applied voltage, and the distance between the nozzle and the collector. This is because the residual solution and the resin deposited on the collector 3 are charged by themselves and repel with each other, and this impede the deposition in a thickness direction. In this connection, the fibrous resin derived from the solution deposits on the collector 3 while evaporating most of the solvent, but a trace amount of the solution deposits as intact on the collector 3.

In contrast, according to the, embodiment of the present invention with reference to FIG. 2, a three-dimensional steric structure can be formed by carrying out electrospinning while grounding the nozzle of the syringe 2 without applying a charge thereto, and, contrarily, applying a positive charge to the collector 3. According this technique, if a regular spinning solution is slowly extruded from the tip of the nozzle, the spinning solution falls as droplets, because the solution is not charged. However, when the spinning solution further contains a liquid, such as water, having a greater relative dielectric constant than that of the biodegradable resin, the liquid is affected by the electric field, and the spinning solution may be drawn toward the collector by the action of polarization. In this case, the spinning solution is not charged by itself and readily three-dimensionally deposits on the collector 3 without suffering from electrostatic repulsion. In this process, the liquid (solution) is divided to two or more strands and drawn from the nozzle of the syringe 2 toward the collector 3, and these strands are entangled to form a flocculent three-dimensional steric structure on the collector 3. To allow this phenomenon to occur, however, the spinning solution should have a somewhat low viscosity. The spinning solution, if having an excessively high viscosity, may not reach the collector 3 even when affected by the electric field. Accordingly, the diameter of the fibrous substance constituting the three-dimensional steric structure prepared according to the present embodiment is substantially controlled by the viscosity of the spinning solution. When the spinning solution has a particularly low viscosity, the fibrous substance more easily deposits to form a three-dimensional steric structure and more easily has a smaller fiber diameter. Typically, when the spinning solution is prepared by dissolving a PLA in chloroform to give a solution and adding ethanol and water thereto, the resulting fibrous substance has a fiber diameter of 0.05 μm or more and less than 10 μm. It is acceptable to apply not a positive charge but a negative charge to the collector 3, as long as the spinning solution is drawn toward the collector by the action of polarization.

The resulting three-dimensional steric structure is cut into a piece of necessary size, and the cut piece is soaked in a buffer solution containing calcium ions and phosphate ions and being saturated with respect to hydroxyapatite to coat the surface of its fibrous skeleton with a hydroxyapatite easily. Examples of the buffer solution for use herein include a Tris buffer solution (pH 7.2 to 7.4) (SBF) containing ions in a concentration substantially equal to the inorganic ion concentration in human plasma; and a solution (1.5 SBF) containing ions in concentrations 1.5 times those of SBF. The 1.5 SBF is more advantageous, because the fibrous substance can be coated with a hydroxyapatite more rapidly.

According to the present embodiment, there is provided a flexible material for filling bone defects, which material has a three-dimensional steric structure including a fibrous substance, in which the fibrous substance contains a biodegradable resin, represented by a poly (lactic acid) (PLA), as a principal component and further contains or bears a siloxane. There is also provided a filling material for bone-repairing, in which the surface of the fibrous substance constituting the three-dimensional steric structure is coated with a hydroxyapatite. The material surely including a communicating space for the entrance of cells and having improved fittability in the affected area can be easily prepared by adopting the technique for producing a nonwoven fabric through electrospinning to the production of the three-dimensional steric structure. In addition, the coating with an absorbable hydroxyapatite can be easily performed by soaking the electrospun article in a buffer solution being supersaturated with respect to hydroxyapatite, and the coated absorbable hydroxyapatite helps to provide higher initial cellular adhesion.

The material for filling bone defects thus obtained has satisfactory flexibility derived from the three-dimensional steric structure constituted by the fibrous substance, shows high cell proliferation in cellular affinity assay using osteoblast-like cells (MC3T3-E1 cells), and excels in bone reconstruction ability.

### EXAMPLES

The present invention will be illustrated in further detail with reference to several examples below which relate to production methods of three-dimensional steric structures. It should be noted, however, that these examples are illustrated only by way of example for understanding the present invention more deeply and are never intended to limit the scope of the present invention.

### Raw Materials Used in Examples

Poly(lactic acid) (PLA): PURASORB PL Poly(L-lactide), having a molecular weight of from 20×10⁴ to 30×10⁴, PURAC biochem (a division of CSM; Netherlands)

Chloroform (CHCl₃): analytical grade reagent, with a purity of 99.0% or more, Kishida Chemical Co., Ltd., Japan

γ-Aminopropyltriethoxysilane (APTES): (TSL8331, with a purity of 98% or more, GE Toshiba Silicones Co., Ltd., Japan)

Siloxane-doped calcium carbonate (Si—CaCO₃): Vaterite containing a siloxane in terms of a silicon ion content of 2.9 percent by weight and prepared by using slaked lime (Microstar T; with a purity of 96% or more; Yabashi Industries Co., Ltd., Japan), methanol (analytical grade reagent; with a purity of 99.8% or more; Kishida Chemical Co., Ltd., Japan), APTES, and carbon dioxide gas (high-purity liquefied carbon dioxide gas; with a purity of 99.9%; Taiyo Kagaku Kogyo K.K., Japan)

### Electrospinning Conditions in Examples

Spinning solution feed rate: 0.1 ml/min,

Applied voltage: A voltage was applied to the plate collector at 25 kV, while the nozzle being grounded,

Distance between the nozzle and the plate collector: 100 mm,

Spinning time: about 60 minutes

### Example 1

APTES (1 g) was added to ultrapure water (0.5 g) with stirring to give a solution. The solution was added dropwise to a 8 percent by weight PLA solution in CHCl₃ so as to give APTES contents of 0.015 g and 0.050 g, respectively, followed by stirring. During this procedure, APTES was condensed to give a siloxane. To the resulting mixtures were added 1.5 g of ethanol and 1 g of ultrapure water to give spinning solutions. These spinning solutions were subjected to electrospinning and thereby yielded three-dimensional steric structures each including a fibrous substance containing a biodegradable resin as a principal component and containing or bearing a siloxane (hereinafter these steric structures are referred to as Si-PLA₁₅ and Si-PLA₅₀, respectively).

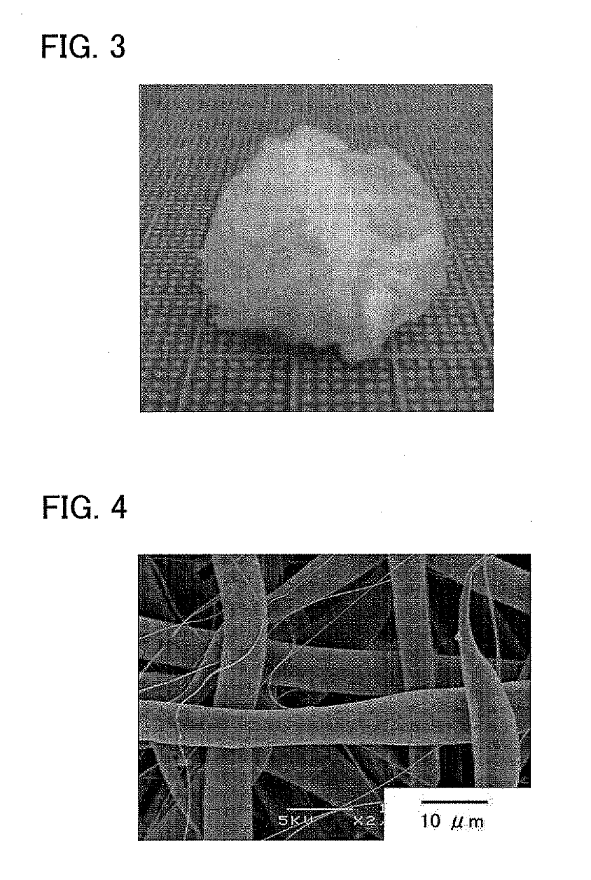

FIG. 3 depicts the appearance of the resulting three-dimensional steric structure (Si-PLA₁₅). FIG. 4 is a scanning electron micrograph (SEM) of this steric structure, demonstrating that the steric structure is a flocculent structure including fibers having diameters of from several tens of nanometers to eight micrometers. The structures under this condition had a weight of 40 mg. The structures did not lose their flexibility and elasticity even after they were soaked in a cell culture medium and recovered therefrom.

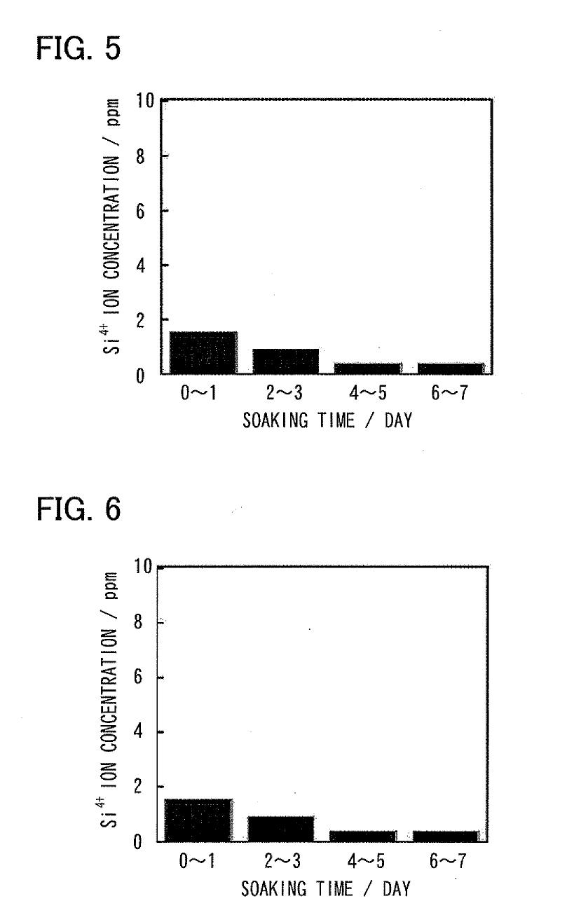

Each of the above-prepared steric structures was cut to a piece 10 mm wide, 10 mm long, and 1 mm thick, soaked in 4 ml of cell culture medium α-MEM, held at a temperature of 37° C. in an incubator in an atmosphere of 5% carbon dioxide gas, and the cell culture medium was exchanged with fresh one on Day 1, Day 3, and Day 5. FIG. 5 and FIG. 6 show the release amounts of silicon ions when the steric structures were soaked in the cell culture medium as measured through inductively coupled plasma emission spectrometry. The data demonstrate that both the samples (steric structures) released a large amount of silicon ions on Day 1, and thereafter released silicon ions in a significantly decreased amount, but continued to release silicon ions at least up to Day 7. Si-PLA₅₀ released about 6.5 ppm of silicon ions on Day 1 but released 1 ppm or less of silicon ions from Day 6 to Day 7, showing only a slight difference from that in Si-PLA₁₅.

### Example 2

A Si—CaCO₃/PLA three-dimensional steric structure was prepared by kneading PLA and Si—CaCO₃ in a heating kneader at 200° C. for 15 minutes to give a Si—CaCO₃/PLA composite containing 40 percent by weight of Si—CaCO₃; mixing 1.67 g of the Si—CaCO₃/PLA composite with 8.33 g of CHCl₃ to give a solution; adding 1.5 g of ethanol and 1 g of ultrapure water to the solution to give a spinning solution; and subjecting the spinning solution to electrospinning under the above-mentioned conditions.

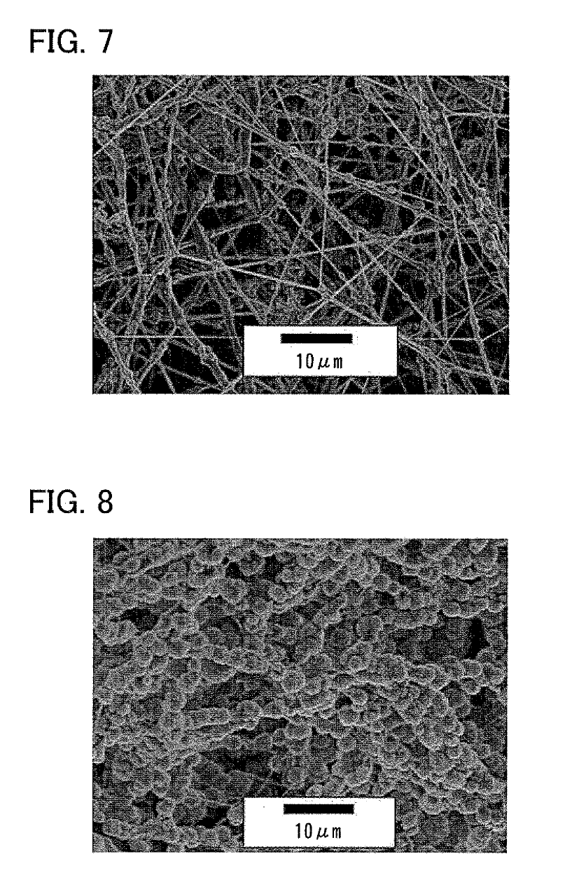

The prepared three-dimensional static structure has a flocculant appearance substantially the same as that shown in FIG. 3 and had superior flexibility and elasticity. FIG. 7 is a scanning electron micrograph (SEM) of the Si—CaCO₃/PLA three-dimensional steric structure, demonstrating that this steric structure is a structure including fine fibers having diameters of about 0.1 to about 3 μm, and spherical calcium carbonate particles having diameters of about 1 μm embedded between the fibers. The fibers have small diameters, and spaces (clearances) between fibers are sufficiently large of about several tens of micrometers or more so as to give sufficient spaces to allow cells to enter therein. The release amount of silicon ions from this steric structure was determined by the procedure of Example 1 to find that the steric structure released silicon ions in amounts of 5.3 ppm on Day 1, 0.8 ppm from Day 2 to Day 3; 0.4 ppm from Day 4 to Day 5; and 0.4 ppm from Day 6 to Day 7, indicating that the release in trace amount of silicon ion continued.

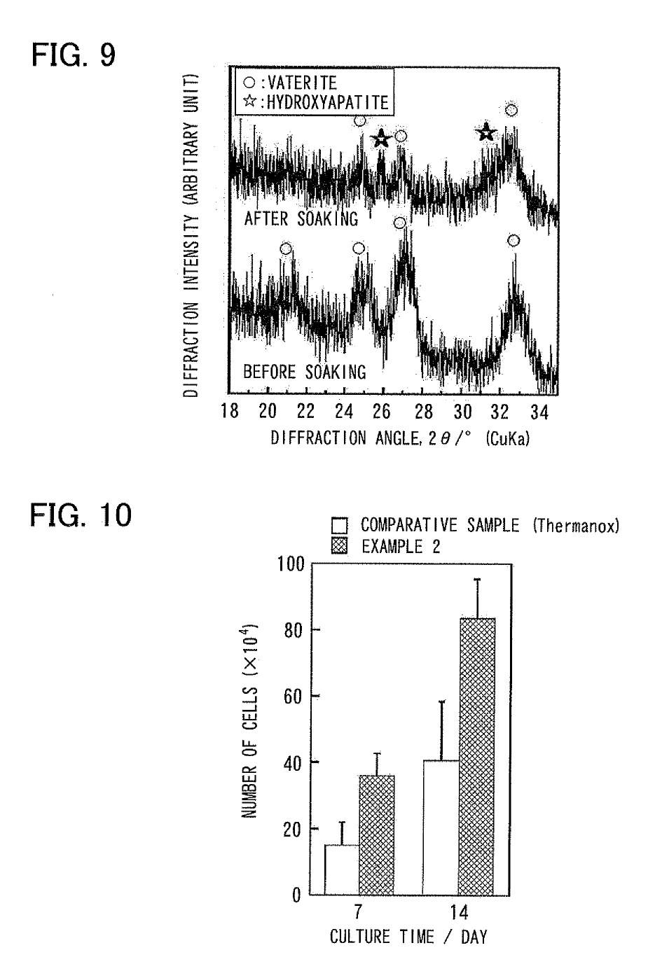

The steric structure was cut to a sample piece 10 mm wide 10 mm long and 10 mm thick, soaked in 40 ml of 1.5 SBF, and held at 37° C. for one day. The sample piece was thereafter recovered from 1.5 SBF and observed under a scanning electron microscope (SEM), to find that a large number of aggregated particles as shown in FIG. 8 precipitated and that there remained spaces of about several tens of micrometers so as to allow cells to enter therein. FIG. 9 shows X-ray diffraction patterns of the sample piece before and after soaking in the 1.5 SBF, demonstrating that peaks derived from hydroxyapatite were observed in the sample piece after the soaking. These results demonstrate that the surfaces of fibers constituting the Si—CaCO₃/PLA three-dimensional steric structure can be easily coated with a hydroxyapatite merely by soaking the steric structure in 1.5 SBF.

FIG. 10 shows how cellular numbers (in terms of cellular numbers per 1 cm²) vary after the inoculation of murine osteoblast-like cells (MC3T3-E1) on the hydroxyapatite-coated steric structure and on a comparative sample (Thermanox: plastic disc for cell culture). The comparative sample Thermanox had been treated on its surface for improving cell proliferation and for use in cell culture. The data in FIG. 10 demonstrate that the steric structure gives much higher cell growth capability than that of the surface-treated comparative sample and is expected as a material which excels in bone reconstruction ability.

### Conditions for Cell Culture Experiment

### Cultivation:

Cultivation using 24-well plate,

Cell type: murine osteoblast-like cells (MC3T3-E1 cells; Riken Institute of Physical and Chemical Research, Japan)

Cell inoculation number: 1×10⁴ cells/well

Medium: α-MEM (containing 10% fetal bovine serum)

Medium exchange: on the day following the inoculation, thereafter every other day

Sample piece: The sample steric structure was cut to a piece 10 mm long, 10 mm wide, and 10 mm thick (10-mm cube)

Cell counting method: The measurement was performed using the Cell Counting Kit-8 (cellular growth/cellular toxicity analytical reagent; Dojindo Laboratories, Japan) in accordance with the protocol attached to the reagent.

While the above description is of the preferred embodiments of the present invention, it should be appreciated that the invention may be modified, altered, or varied without deviating from the scope and fair meaning of the following claims.

What is claimed is:

1. A material for filling bone defects, the material having a flocculent three-dimensional steric structure comprising a fibrous substance, the fibrous substance containing a biodegradable resin as a principal component and containing or bearing a siloxane.

2. The material according to claim 1, wherein the fibrous substance has a diameter of 0.05 μm or more and less than 10 μm.

3. The material according to claim 1, wherein the fibrous substance is coated with a hydroxyapatite on its surface.

4. The material according to claim 2, wherein the fibrous substance is coated with a hydroxyapatite on its surface.

5. The material according to claim 1, wherein the biodegradable resin is a poly (lactic acid) or a copolymer thereof.

6. The material according to claim 2, wherein the biodegradable resin is a poly (lactic acid) or a copolymer thereof.

7. The material according to claim 3, wherein the biodegradable resin is a poly (lactid acid) or a copolymer thereof.

8. The material according to claim 4, wherein the biodegradable resin is a polylactic acid) or a copolymer thereof.

9. The material according to claim 1, wherein the fibrous substance contains or bears the siloxane as dispersed in calcium carbonate microparticles.

10. The material according to claim 2, wherein the fibrous substance contains or bears the siloxane as dispersed in calcium carbonate microparticles.

11. The material according to claim 3, wherein the fibrous substance contains or bears the siloxane as dispersed in calcium carbonate microparticles.

12. The material according to claim 4, wherein the fibrous substance contains or bears the siloxane as dispersed in calcium carbonate microparticles.

13. A method for producing a material for filling bone defects, the method comprising the steps of:
preparing a solution or slurry of a substance by dissolving or suspending the substance in a solvent, the substance containing a biodegradable resin as a principal component and containing or bearing a siloxane; and
carrying out electrospinning of the solution or slurry while applying a charge not to the solution or slurry but to a collector to thereby yield a material for filling bone defects on the collector, the material having a three-dimensional steric structure comprising a fibrous substance, the fibrous substance containing the biodegradable resin as a principal component and containing or bearing the siloxane.

14. The method according to claim 13, wherein the step of preparing the solution or slurry further comprises adding a liquid to the solution or slurry, the liquid having a relative dielectric constant greater than that of the biodegradable resin.

15. The method according to claim 14,
wherein the biodegradable resin is a polylactic acid) and/or a copolymer thereof,
wherein the solvent is chloroform and/or dichloromethane,
wherein the liquid having a greater relative dielectric constant is water, and
wherein the step of preparing the solution or slurry further comprises adding an amphiphilic liquid to the solution or slurry, the amphiphilic liquid being satisfactorily miscible with the solvent and with the water.

16. The method according to claim 15, wherein the amphiphilic liquid is at least one selected from the group consisting of methanol, ethanol, propanol, and acetone.

17. The method according to claim 13, further comprising the step of soaking the electrospun material for filling bone defects in a buffer solution being supersaturated with respect to hydroxyapatite.

18. The method according to claim 14, further comprising the step of soaking the electrospun material for filling bone defects in a buffer solution being supersaturated with respect to hydroxyapatite.

19. The method according to claim 15, further comprising the step of soaking the electrospun material for filling bone defects in a buffer solution being supersaturated with respect to hydroxyapatite.

20. The method according to claim 16, further comprising the step of soaking the electrospun material for filling bone defects in a buffer solution being supersaturated with respect to hydroxyapatite.
